(19) United States
(12) Patent Application Publication
(10) Pub. No.: US 2025/0186455 A1
(43) Pub. Date: Jun. 12, 2025

(54) TREATMENT COMPOSITION FOR TREATING CHRONIC PAIN AND MIXED PAIN SITES

(71) Applicants: Taghogho Agarin, San Jose, CA (US); Sterling Okafor Mgbekwe, Buena Park, CA (US); Robert McLay, Chula Vista, CA (US)

(72) Inventors: Taghogho Agarin, San Jose, CA (US); Sterling Okafor Mgbekwe, Buena Park, CA (US); Robert McLay, Chula Vista, CA (US)

(21) Appl. No.: 18/971,859

(22) Filed: Dec. 6, 2024

Related U.S. Application Data
(60) Provisional application No. 63/607,376, filed on Dec. 7, 2023.

(51) Int. Cl. A61K 31/55; A61K 31/137; A61K 31/167; A61K 31/192; A61K 31/197; A61K 31/381; A61K 31/415; A61K 31/485; A61K 31/5415; A61K 45/06

(52) U.S. Cl. CPC A61K 31/55; A61K 31/137; A61K 31/167; A61K 31/192; A61K 31/197; A61K 31/381; A61K 31/415; A61K 31/485; A61K 31/5415; A61K 45/06

(57) ABSTRACT

A composition for the treatment of chronic pain and mixed pain sites may include a serotonin and norepinephrine reuptake inhibitor; a non-steroidal anti-inflammatory agent; optionally acetaminophen; optionally oxcarbamazepine; optionally an oral opioid antagonist; optionally an antacid; optionally an anti-epileptic; and optionally an opioid. A method for treating chronic pain and mixed pain sites may include administering, to a patient in need, a therapeutically effective dosage of the composition.

### RELATED APPLICATION

This application claims priority to provisional patent application U.S. Ser. No. 63/607,376 filed on Dec. 7, 2023, the entire contents of which is herein incorporated by reference.

### BACKGROUND

The embodiments described herein relate generally to treatment compositions and, more particularly, to a treatment composition for treating chronic pain and mixed pain sites.

Chronic pain is defined as pain on most days or every day in the past 6 months. In 2016, an estimate 20.4% (50.0 million) of US adults had chronic pain and 8.0% (19.6 million) had high-impact chronic pain with higher incidence among women, older adults, adults with public health insurance, and rural residents. Chronic pain is one of the most common reasons adults seek medical care. Chronic pain contributes to an estimated $560 billion each year in direct medical costs, lost productivity, and disability programs. Causes of chronic pain including conditions like diabetes, arthritis, fibromyalgia, irritable bowel, and back pain, among others.

Pain medications are a core component in the management of chronic pain. The most effective management protocols use a combination of prevention, psychological, physical, and pharmaceutical approaches. The treatment goal is to reduce pain, maximize function, and improve the quality of life. Several categories of medication are currently used in chronic pain management. They include non-opioid analgesics, like acetaminophen, nonsteroidal anti-inflammatory drugs, benzodiazepines, opioids, cannabinoids, and topical agents as well as adjuvant mediations originally developed for other purposes but having analgesic properties too.

A mixed pain site is a site on an individual in which there is a complex overlap of the different known pain types (nociceptive, neuropathic, nociplastic) in any combination, acting simultaneously and/or concurrently to cause pain in the same body area. Current treatments for treating mixed pain sites include taking multiple separate medications, wherein each of the separate medications targets a different pain type. Taking multiple medications, however, is not ideal as this requires the administration of multiple medications simultaneously and requires a patient to keep track of which medications have been taken, at what dosages, and when. As such, there is much room for error in the administration of the medications to treat the mixed pain sites.

The use of combination drug therapy to achieve improved pain relief has been known for decades. Opioids are known strong analgesics for the treatment of moderate to severe pain. Their use, however, is sometimes complicated by physiological and psychologic dependence. Since opioid analgesics cause physical and psychological dependence long after the duration of intended treatment, some patients end up satisfying their cravings using illicit opioids, which can lead to fatal overdoses.

Conventionally treatment options for patients with chronic pain and mixed pain sites include switching to different pain medications and use of non-opioid alternatives. However, some of these solo approaches often have limitations, including delayed onset of action, side effects, and inadequate efficacy, hence the combination of certain classes.

Therefore, what is needed is a method and composition for treating chronic pain and mixed pain sites.

### SUMMARY

Some embodiments of the present disclosure include a composition for the treatment of chronic pain and mixed pain sites. The composition may include a serotonin and norepinephrine reuptake inhibitor; a non-steroidal anti-inflammatory agent; optionally acetaminophen; optionally oxcarbamazepine; optionally an oral opioid antagonist; optionally an antacid; optionally an anti-epileptic; and optionally an opioid. A method for treating chronic pain and mixed pain sites may include administering, to a patient in need, a therapeutically effective dosage of the composition.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The composition of the present disclosure may be used as a single administration treatment for treating chronic pain and mixed pain sites and may comprise the following elements. This list of possible constituent elements is intended to be exemplary only, and it is not intended that this list be used to limit the composition of the present application to just these elements. Persons having ordinary skill in the art relevant to the present disclosure may understand there to be equivalent elements that may be substituted within the present disclosure without changing the essential function or operation of the composition.

The various elements of the present disclosure may be related in the following exemplary fashion. It is not intended to limit the scope or nature of the relationships between the various elements, and the following examples are presented as illustrative examples only.

By way of example, some embodiments of the present disclosure include a method and composition for treating chronic pain and mixed pain sites, wherein the method comprising administering, to a patient in need, a therapeutically effective dosage of a composition comprising a serotonin-noradrenaline reuptake inhibitor (SNRI), a non-steroidal anti-inflammatory agent (NSAID), such as ibuprofen, optionally acetaminophen, optionally oxcarbamazepine, and optionally an oral opioid antagonist, wherein a therapeutically effective dosage may be a dosage that, when taken as directed, provides the desired results. In some embodiments, the composition may further comprise an antacid. Embodiments of the composition may also further comprise an anti-epileptic. In yet further embodiments, the composition may further comprise an opioid. In additional embodiments, the composition may further comprise a glycosaminoglycan. The combined dosages may vary in amount and administration, particularly depending on level of pain. In some embodiments, the composition may be provided as a single administration in, for example, a pill form, wherein only a single pill needs to be administered to the patient to provide the therapeutically effective dosage. In other embodiments, the composition may be used as a repeated multiple dose administration. In embodiments, the method and composition of the present disclosure may be used for the treatment of pain following any common pain diagnosis, such as fibromyalgia, neuropathy, chronic regional pain syndrome, and failed back surgery syndrome.

The SNRI may be, for example, duloxetine, its prodrug, and drug polymorphs, and the oral opioid antagonist may be, for example, naltrexone, such as low dose naltrexone. Other suitable SNRIs include venlafaxine, desvenlafaxine, and levominalcipram. Inclusion of the SNRI may help address neuropathic pain. More specifically, duloxetine effectively reduces pain behavior across a range of persistent, neuropathic, and inflammatory pain models. Duloxetine analgesic effect may result from increased activity of serotonin and norepinephrine within the central nervous system. The oral opioid antagonist may help address central pain. Naltrexone is a known opioid receptor antagonist that blocks the effect of opioids and prevents opioid intoxication and physiologic dependence on opioid users. In a low dose, naltrexone may act as a glial/immune cell modulator, decreasing pain by helping reduce inflammation.

In embodiments, including an NSAID in the composition of the present disclosure may help address nociceptive pain. As mentioned above, the NSAID may be ibuprofen. However, other suitable NSAIDs may comprise naproxen, diclofenac sodium, celecoxib, refecoxib, valdecoxib, meloxicam, piroxicam, indomethacin, sulindac, ketorolac, nabumetone, etodolac, ketoprofen, fenoprofen, flurbiprofen, mefenamic acid, oxaprozin, aspirin, diflunisal, salsalate, and the like and any prodrug and drug polymorphs thereof. In yet further embodiments, the composition may further comprise a weak inhibitor of cyclooxygenase I and II, such as paracetamol and salicyclase.

As mentioned above, the composition of the present disclosure may comprise an SNRI and an opioid antagonist, such as naltrexone. In embodiments, such as when the condition being treated is spondylosis, spondylolysis, spondyloarthropathy, spondylolisthesis, spondyloarthritis, or spondylitis, or failed surgery back syndrome, the composition may further comprise a non-steroidal anti-inflammatory agent, such as ibuprofen, and acetaminophen, which may inhibit the cyclooxygenase pathway. Additionally or alternatively, oxcarbamazepine may be added in place of or in addition to the acetaminophen. These additional ingredients may be added for treating increased levels of pain, such as during the first 2-3 weeks after a surgery or diagnosis.

In some instances, the combination of an SNRI, such as duloxetine, and an NSAID, such as ibuprofen, may increase the risk of gastrointestinal bleeding. As such, to offset this risk, the composition may further comprise an antacid.

As mentioned above, the composition may also include an anti-epileptic. Suitable anti-epileptics include pregabalin, which may help treat nerve and muscle pain. As also mentioned above, the composition may further comprise an opioid, such as buprenorphine, which may help treat pain.

As also mentioned above, the composition may also include a glycosaminoglycan. In embodiments, the glycosaminoglycan may be, for example, glucosamine sulfate or chondroitin sulfate.

For each of the components listed above, it is noted that the component may be the component, its prodrug, or drug polymorph.

In embodiments, the composition of the present disclosure may comprise, for example, about 1.0 to about 5.0 weight % (wt. %) oral opioid antagonist, such as low-dose naltrexone, and about 0.1 to about 60 wt. % SNRI. For example, in a particular embodiment, the composition may comprise form about 1 to 1000 mg NSAID, from about 0.5 mg to about 5 mg naltrexone, and about 1 to about 350 mg venlafaxine. When included, the amount of the glycosaminoglycan may be from about 1 to about 1500 mg. The composition may be administered in a daily formulation to prevent or reduce pain, as measured by the graded chronic pain scale revised (GCPS-R).

The combination of low dose naltrexone (LDN) and duloxetine in a single pill composition may prevent disease progression and modify the course of chronic pain, delay/prevent relapse, or recurrence of chronic pain. The treatment may also provide a more effective treatment than conventional treatments and may increase the response rate to treatment. In fact, combining LDN and duloxetine into a single pill may result in a synergistic combination, wherein the combination works better than administering the ingredients independently to the patient in need. Specifically, the composition of the present disclosure may reduce nociceptive, neuropathic, and/or central pain states.

The composition may be formed into a single administration composition, which may be manufactured using a mixture of a tablet or extended-release injectable suspension for naltrexone and a tablet or oral suspension for duloxetine mixed together and provided as a single tablet, capsule, oral disintegrating tablet, oral disintegrating strip, oral dispersible films, liquid solutions, extended-release preparations, controlled release forms, lozenges, films, nasal sprays, patches, and the like. The single administration may result in only a single administration form, such as a single tablet, needing to be administered to a user to provide a therapeutically effective dosage. The composition may be in the form of a single long-acting depot injection given individually or combined that is gradually released over days, weeks, or even months. The individual or combined medications may also be administered as an implant placed under the skin.

While the above descriptions indicates that the composition may be administered as a single pill, the composition is not limited to such administration. Rather, the composition may be administered in tablets, capsules, oral disintegrating tablets, oral disintegrating strips, oral dispersible films, liquid solutions, extended-release preparations, controlled release forms, lozenges, films, nasal sprays, patches or topical cream applications. Moreover, the composition may be provided in a therapeutically effective dosage, which may be administered on-demand or in set, pre-determined periods of time.

A method of treating chronic pain and mixed pain sites in a patient may comprise administering, to a patient in need, a therapeutically effective dosage of the composition of the present disclosure.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

What is claimed is:

1. A composition for the treatment of chronic pain and mixed pain site, the composition comprising:
a serotonin and norepinephrine reuptake inhibitor (SNRI); and
a non-steroidal anti-inflammatory agent (NSAID).

2. The composition of claim 1, further comprising an oral opioid antagonist.

3. The composition of claim 2, wherein:
the SNRI is duloxetine, duloxetine prodrug, or a duloxetine drug polymorph;
the NSAID is a member selected from the group consisting of ibuprofen, celecoxib, and meloxicam or a prodrug or drug polymorph of ibuprofen, celecoxib, or meloxicam; and
the oral opioid antagonist is naltrexone, naltrexone prodrug, or a naltrexone drug polymorph.

4. The composition of claim 2, further comprising an antacid.

5. The composition of claim 2, further comprising acetaminophen.

6. The composition of claim 2, further comprising oxcarbamazepine.

7. The composition of claim 2, further comprising an anti-epileptic.

8. The composition of claim 7, wherein the anti-epileptic is pregabalin.

9. The composition of claim 2, wherein the composition comprises:
about 5.0 wt. % oral opioid antagonist; and
about 0.1 to about 60 wt. % SNRI.

10. The composition of claim 2, wherein:
the oral opioid antagonist is naltrexone;
the SNRI is venlafaxine; and
a therapeutically effective dosage of the composition comprises:
about 1 to about 1000 mg NSAID;
about 0.5 mg to about 5 mg naltrexone; and
about 1 to about 350 mg venlafaxine.

11. The composition of claim 1, wherein the composition is in a single administrable form.

12. A method for treating chronic pain and mixed pain sites, the method comprising:
administering a therapeutically effective dosage of a composition to a patient in need,
wherein:
the composition comprises:
a serotonin and norepinephrine reuptake inhibitor (SNRI); and
a non-steroidal anti-inflammatory agent (NSAID).

13. The method of claim 12, wherein the composition further comprises an oral opioid antagonist.

14. The method of claim 13, wherein:
the SNRI is duloxetine, duloxetine prodrug, or a duloxetine drug polymorph;
the NSAID is a member selected from the group consisting of ibuprofen, celecoxib, and meloxicam or a prodrug or drug polymorph of ibuprofen, celecoxib, or meloxicam; and
the oral opioid antagonist is naltrexone, naltrexone prodrug, or a naltrexone drug polymorph.
